(19) United States
(12) Patent Application Publication
(10) Pub. No.: US 2021/0322945 A1
(43) Pub. Date: Oct. 21, 2021

(54) DEVICE AND METHOD FOR PLASMA ACTIVATION OF A LIQUID

(71) Applicant: Erbe Elektromedizin GmbH, Tuebingen (DE)

(72) Inventors: Kristin Brunecker, Rottenburg (DE); Alexander Neugebauer, Moessingen (DE)

(73) Assignee: Erbe Elektromedizin GmbH, Tuebingen (DE)

(21) Appl. No.: 17/229,473

(22) Filed: Apr. 13, 2021

(30) Foreign Application Priority Data
Apr. 16, 2020 (EP) 20169750.5

(51) Int. Cl. B01J 19/08; B01J 19/00

(52) U.S. Cl. CPC B01J 19/08; B01J 19/0033; B01J 2219/0894; B01J 2219/0801; B01J 2219/0875; B01J 2219/00164

(57) ABSTRACT

A device for creation of a plasma-activated liquid with defined characteristics. The device includes a plasma application device having a plasma applicator. A liquid is brought into contact with a gas plasma in the plasma application device. A sensor device serves for analysis of the composition of the plasma-treated liquid at least in terms of a species created by the plasma treatment. Based on a concentration of one or more species detected by the sensor device, treatment parameters of the liquid in the plasma application device can be adjusted or modified. Thereby a plasma-treated liquid with defined characteristics for treatment of a patient is provided.

### CROSS REFERENCE TO RELATED APPLICATION

This application claims priority to European Application No. 20169750.5, filed Apr. 16, 2020, the entirety of which is incorporated by reference herein.

### BACKGROUND

Embodiments of the invention include a device and a method for plasma activation of a liquid for application on a patient.

Due to the influence of a plasma on a liquid, such as for example NaCl-solution, Ringer's lactate solution or another, this liquid can be transferred in a plasma-activated condition. Then the liquid comprises reactive species, e.g. radicals with increased Redox-potential, as well as substances such as peroxides, peroxide nitrites, nitrates, nitrites, nitrogen oxides (oxide or nitrogen-centered reactive species), that comprise a medical efficacy. Such activated liquids can be used for an indirect plasma treatment. The effect on the tissue to be treated can be a disinfecting effect. Microbial and bacterial organisms can be deactivated by the liquid. In addition, the treatment can be based on that the plasma-activated liquid has different effects on pathogen and healthy cells. The different effect is based on the species contained in the plasma-activated liquid.

For creation of plasma-activated liquids WO 2015/123720 A1 proposes application of a plasma on a gel surface. Reference is particularly made on non-thermal so-called cold plasmas that comprise a highly active mixture of oxygen, nitrogen and hydrogen radicals, ions, electrons, photons and ultraviolet radiation.

US 2012/0111721 A1 and WO 2007/117634 A2 propose a device for plasma treatment of a liquid. The plasma treatment is carried out in a rotational symmetric vessel on the wall of which a thin liquid film flows. Thereby the discharge occurs between two graphite electrodes, whereby the created radiation influences the liquid.

EP 1 702 678 A1 proposes the treatment of a liquid film with electron radiation in a vacuum vessel. Alternatively, in this document the treatment of a liquid film with a UV-source is considered that is provided instead of or in addition to the electron radiation source.

EP 2 937 103 A1 proposes the activation of an action medium, e.g. a wound pad, by means of plasma. For this the wound pad can comprise a gel that can be plasma-activated, a liquid that can be plasma-activated or a fluid-impregnated carrier layer that can be plasma-activated. By means of plasma treatment of the action medium, an active substance is created or released therein. Water-based liquids, saline solutions and gels derived therefrom are considered as action media. For plasma treatment a plasma generator is provided that creates a cold plasma with atmospheric pressure, for example. This plasma is applied on the action medium in a two-dimensional manner. After activation the wound pad can be attached on the patient.

US 2019/0110933 A1 and WO 2017/167748 A1 propose a wound treatment by a combined plasma and vacuum application. For this a vacuum therapy device is connected with an anti-microbacterial and healing atmospheric plasma source, wherein a sensor device can be provided for detection of the healing. For this the sensor system can detect at least one physical parameter on the body surface of the patient. Based on this detected parameter, the condition of the wound is evaluated. Detected parameters can be particularly the temperature, the pressure, the humidity and/or pH-value.

The problem of deterioration of the medical effect of plasma-activated liquids during longer storage is known from WO 2017/074979 A1. In order to maintain such plasma-activated liquids for a longer term in a storable condition, it is proposed to use liquids for plasma activation, the content of cysteine, methionine, phenylalanine and phenol red thereof is reduced and to which three-nitro-L-tyrosine is added.

US 2014/0322096 A1 and WO 2014/055812 A1 describe a disinfection system, particularly for hand disinfection of surgeons. For this a reaction vessel is provided in which a barrier discharge occurs between two insulated electrodes. An atomized washing liquid is conveyed through the plasma created thereby that is collected on the bottom in the vessel and can then be dispensed for hand disinfection via nozzles.

US 2015/0306258 A1 describes a method for sterilization of cornea tissue prior to its transplantation. For this the tissue is subject to a plasma-activated fluid. For a plasma activation the fluid is conveyed in atomized condition prior to the application on the cornea as spray cone through a cold plasma discharge.

WO 2017/091534 A1 describes methods and a system for killing or deactivation of spores by application of a liquid on the surface to be sterilized, wherein the liquid comprises an additive in order to maintain the plasma activation at least over a time period of 30 seconds. The additive can be for example a nitrite, a bio-active oil, an acid, a transition metal or an enzyme.

Contrary to this, WO 2014/145570 A1 and WO 2014/152256 A1 propose direct application of a plasma on a water surface, whereby radicals are created in the water. The plasma-activated water can be used for surface disinfection.

WO 2017/083323 A1 also addresses the creation of a plasma-activated liquid. For this a device is provided in which an aerosol consisting of gas and liquid is passed along one or more plasma generators in order to activate the gas and/or the liquid. For the separation of gas and liquid after plasma treatment a precipitation device is provided. The liquid can be used for disinfection purposes. A barrier discharge serves for plasma creation.

WO 2018/089577 A1 addresses systems and methods for plasma activation of liquids, wherein the activation shall achieve particularly high concentrations and wherein large liquid amounts shall be activated. For this a device is provided that transfers the liquid in a thin layer, wherein the plasma is created nearby the thin liquid layer.

The plasma activation of a liquid of a discharge is known from US 2019/0279849 A1, wherein the current flow leads from an electrode to the liquid. Distances of 6 mm to 10 mm are proposed. In addition, a light-receiving device is provided in order to detect typical lines of created radicals or ions in the context of the optical emission spectroscopy.

CN 109121278 A also operates with the direct influence of an electrical discharge on a liquid and current flow therethrough. The discharge can also be an electrical barrier discharge.

WO 2017/192618 A1 describes a device and method for creation for a plasma-activated aqueous chemotherapeutic agent. For this a discharge vessel is provided in which at least one hollow needle electrode is arranged, wherein the vessel is lined inside in an electrically insulating manner. The electrodes are attached on a rotating hollow shaft into which air is conveyed. A barrier discharge is created between the electrodes and the vessel wall. Liquid conveyed into the vessel gets in firm contact with the barrier discharge and is discharged in an activated condition at the bottom end of the vessel.

With the cited methods, plasma-activated aqueous liquids can be created. It is also known that such activated liquids are at least storable for a limited term. The activated liquids thereby act in a broad spectrum, wherein their efficacy changes over time. This can constitute an uncertainty factor for the application of plasma-activated liquids.

### SUMMARY

Starting therefrom it is the object of embodiments of the invention to improve the reproducibility of the treatment success during medical application of plasma-activated liquids.

An embodiment of the inventive device for supply of a medical instrument with a plasma-activated liquid comprises a plasma generator and a plasma application device by means of which the plasma created by the plasma generator is brought into contact with the liquid. The liquid can be discharged continuously or in portions via an outlet of the plasma application device and can be supplied to an instrument. Thereby the liquid can be discharged directly to the instrument or can be temporarily stored over a period of time in a reservoir vessel.

According to an embodiment of the invention, a sensor device for detection of at least one chemical or physical parameter of the liquid during and/or after the plasma exposure is provided. The parameter detected by the sensor device is used by a control device for control of the characteristics of the plasma-activated liquid discharged to the instrument. The control device comprises an input that is connected with the sensor device. On the output side the control device can be connected with the plasma generator, for example, in order to influence parameters of the plasma discharge. In addition or as an alternative, the control device can be connected with the plasma application device, pumps, valves or similar control elements in order to influence parameters of the plasma treatment. As such, for example, the residence time of the liquid in or at the plasma. If the liquid is brought into contact with the plasma as thin film or as droplets, the control device can influence the flow velocity of the liquid, the droplet size of the liquid, the layer thickness of the liquid and the like. In addition or as an alternative, the control device can be connected with control elements of a reservoir vessel, such as, for example, valves or pumps, in order to influence the residence time of the activated liquid in the reservoir vessel.

Preferably, the plasma generator comprises a gas channel and at least one electrode being in contact with gas from the gas channel. The electrode can be an insulated electrode in order to feed a barrier discharge. It can be an electrode with electrical conducting surface in order to allow a direct transition of electrons from the electrode in the plasma. The gas channel can be applied with a gas, particularly an inert gas, such as argon. In this manner an ionization of the gas and thus the creation of the plasma, particularly a plasma jet that is in contact with the liquid, can occur at the electrode. The liquid can be in electrical contact with an electrode. This is preferably a non-insulated electrode. In this case, it comprises an electrically conductive surface, such that a transfer of electrons from the liquid in the electrode and from the electrode in the liquid is possible. The liquid activation by plasma occurs preferably due to application of the plasma jet on the liquid. Thereby the liquid can be provided as compact body with a, for example, horizontal liquid surface. The plasma jet can also be introduced in the liquid, such that parts of the plasma rise as bubbles in the liquid. In addition, atomized liquid can be introduced in the argon plasma jet. Instead of the argon plasma jet it is, however, also possible to use other types of plasma, particularly air plasma. The plasma can be a cold plasma or also a plasma the gas temperature of which is “warm”, i.e. the gas temperature of which is above the body temperature of humans.

A plasma generator having a non-insulated electrode that allows a current flow through the liquid is particularly suitable for the plasma activation of the liquid.

Due to the plasma treatment, species are created in the liquid, i.e. chemical compounds with different lifetime and reactivity that are in total denoted as plasma-created substances. Such plasma-created substances can be hydronium ions, hydroxide ions, hydrogen peroxide and/or nitrite ions and/or nitrate ions and/or peroxynitrite and/or singlet oxygen and/or ozone and/or oxygen and/or superoxide radical ions and/or hydroxyl radicals and/or hydroperoxyl radicals and/or nitrogen oxides such as nitrogen monoxide and nitrogen dioxide. The sensor device provided for the detection of such species can be a sensor device that is suitable for optical spectroscopy, e.g. for absorption spectroscopy, for ultraviolet light, visible light or infrared light. In addition or as an alternative, the sensor device can be a device suitable for the detection of the phosphorescence, particularly the near infrared phosphorescence. In addition or as an alternative, the sensor device can be a device for carrying out optical emission spectroscopy for UV-light, visible light and/or near infrared light. In addition or as an alternative, the sensor device can be an electron spin resonance spectroscopy device.

### BRIEF DESCRIPTION OF THE DRAWINGS

Further details of advantageous embodiments of the invention are subject of the specification or the drawings. The drawings show:

FIG. 1 shows a block diagram for illustration of structural and functional elements of an embodiment of the inventive device for supply of a medical element with a plasma-activated liquid, and

FIG. 2 shows a plasma application device for creation of plasma-activated liquids.

### DETAILED DESCRIPTION

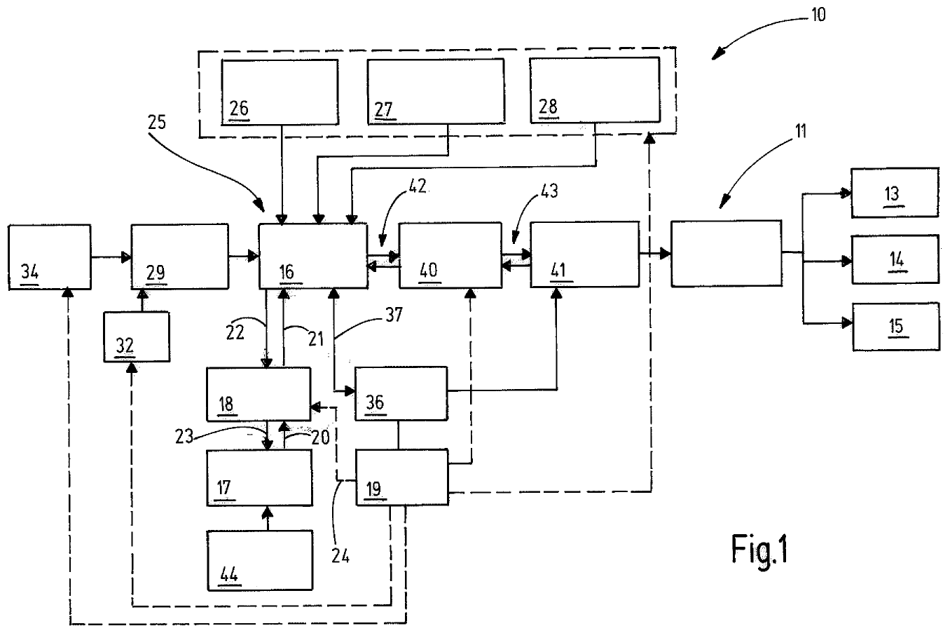

FIG. 1 illustrates a device 10 for supply of a medical instrument 11 with plasma-activated liquid with which at least one of different treatment methods can be carried out. Treatment methods are illustrated by function blocks in FIG. 1, wherein block 13 typifies sub-tissue injection with plasma-activated liquid. Block 14 typifies the alternating treatment of tissue with plasma, e.g. argon plasma, and/or with RF-current as well as plasma-activated liquid. Block 15 typifies the wetting or spraying of tissue with plasma-activated liquid.

An instrument 11 means any instrument with which at least one of the indicated methods illustrated in blocks 13-15 can be executed as well as other instruments for different applications of plasma-activated liquid on a patient or on biological tissue.

The device 10 comprises a treatment vessel 16 in which a liquid, e.g. sodium chloride solution, Ringer's solution, Ringer's lactate solution or another liquid that can be applied on a patient, is to be treated with plasma. The liquid originates from a reservoir vessel 17 that is connected with the treatment vessel 16 via a controllable pump 18. The pump 18 serves to pump liquid out of the reservoir vessel 17 into the treatment vessel 16 under control of the control device 19, as indicated by arrows 20, 21. In addition, the pump 18 can be configured at least as an option to pump liquid back from the treatment vessel 16 into the reservoir vessel 17, as indicated by arrows 22, 23. The feed direction and as required also the feed rate with which the pump 18 operates is set by the control device 19, as indicated by arrow 24.

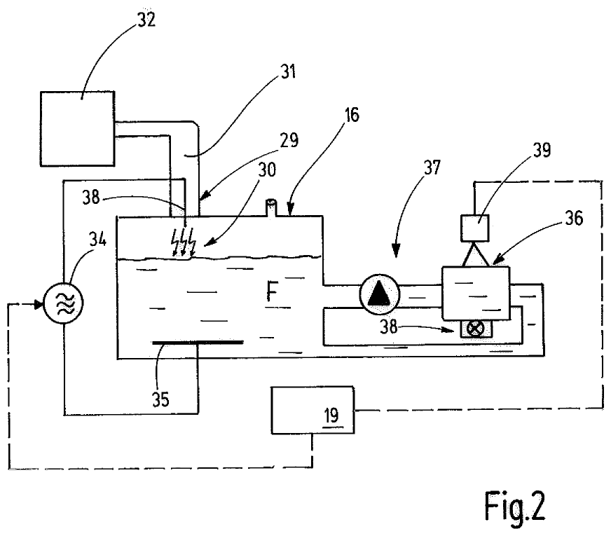

The treatment vessel 16 can be a vessel in which the liquid F to be treated is provided as compact liquid body having a substantially horizontal surface, as illustrated in FIG. 2 in a sketchy manner. Also any other vessel can be used as treatment vessel 16 in which liquid can be brought into contact with a plasma. Such vessels are, e.g. pouring vessels in which a droplet curtain can be poured through a plasma or a liquid layer can be poured along a plasma, vessels having an atomizer that introduce the liquid as spray in a plasma, spinning devices that bring the liquid in form of a thin film in contact with the plasma or the like more. The treatment vessel 16 together with a plasma 30 created therein (FIG. 2) form a plasma application device 25.

For this FIG. 1 illustrates in function blocks 26-28 surrounded by dashes different mixing devices, at least one of which is at least preferably provided and that serve to distribute reactive species created in the treatment vessel homogeneously. A convector 26 can serve for this purpose that creates a convection flow by application of heat or cold on the liquid. As an alternative, a stirring device 27 can be provided that serves to bring the liquid F in the treatment vessel 16 in movement. A function block 28 illustrates a gas swirling device in order to bring the liquid in the treatment vessel in firm contact with the plasma 30. Each device symbolized by the function blocks 26, 27, 28 can be arranged individually or in combination with one or more of the indicated devices in or at the treatment vessel 16.

As apparent from FIG. 2, a plasma applicator 29 is assigned to the treatment vessel 16 that is configured for creation of a plasma 30. For example, the plasma applicator 29 can be an argon plasma applicator. It comprises a gas supply channel 31 via which an inert gas, e.g. nitrogen, a noble gas, e.g. argon or helium, or another gas provided for plasma creation, e.g. a reactive gas, e.g. an oxygen-containing gas, is supplied. The gas channel 31 is connected to a gas source 32. It can be configured in a controllable manner, as indicated in FIG. 1 and can be connected to the control device 19 in order to be controlled in terms of the point of time of the gas discharge and/or in terms of the amount of the gas flow.

The plasma applicator further comprises an electrode 33 configured in a non-insulated manner, i.e. an electrode 33 having an electrically conductive surface that is in contact with or surrounded by the gas flow of the gas supply channel 31.

The electrode is connected to a pole of a plasma generator 34, the other pole of which is, for example, connected with an electrode 35 surrounded by flow of liquid F. The plasma generator is preferably an RF-generator that is configured for supply of a radio frequency voltage and a radio frequency current. The generator is preferably controllable in terms of the amount of the supplied voltage and/or the supplied power and/or the current and/or in terms of the wave form, the modulation of the duty cycle, the crest factor or other parameters. For this the plasma generator 34 can be connected with the control device 19 and can be controlled by it, as illustrated in FIG. 1.

The device 10 further comprises a sensor device 36 that is configured and serves to carry out the analysis of the species formed in the treated liquid F. These species are substances that are formed by a plasma treatment of the liquid F in the broadest sense, also ions, radicals, fractions of molecules and the like. The sensor device 36 can be arranged outside of the treatment vessel 16, as schematically illustrated in FIG. 1 and can be supplied, for example, via a circulation 37 with liquid F from the treatment vessel 16.

A sensor device 36 for optical absorption spectroscopy is schematically illustrated in FIG. 2. For this the sensor device 36 comprises a light emitting device 38 and a light receiving device 39. The light emitting device 38 is, for example, configured to emit ultraviolet, visible and/or infrared light with a known spectral composition into the liquid F. The light receiving device 39 is preferably configured to detect the spectral composition of the ultraviolet, visible and/or infrared light that has passed through the liquid F. The control device 19 is configured to determine the presence and concentration of selected species from the difference between the spectrum of the light emitted by the light emitting device 38 and the light received by the light receiving device 39.

Instead of the sensor device 36 configured for optical absorption spectroscopy, also any other sensor device can be provided that is configured to detect the presence and/or concentration of one or more species in the liquid. Such sensor devices can be emission spectroscopy devices for ultraviolet, visible and/or infrared light. The sensor device can also be a phosphorescence detection device for ultraviolet, visible and/or infrared light, particularly for near infrared. The sensor device 36 can also be a device for pH-measurement, particularly a single rod measuring cell. The sensor device 36 can comprise one or multiple of the above-mentioned sensor devices.

The control device 19 can control the plasma generator 34 in order to control the concentration and/or composition of different species in the liquid F. For example, the control device 19 can control the current and/or voltage supplied by the plasma generator 34 in terms of power, amount, wave form, crest factor, frequency, modulation and the like in order to control the plasma and thereby the creation of species.

The treatment vessel 16 can be connected directly with the instrument 11 via a pump 40 in order to supply the instrument 11 with plasma-activated liquid. As an option, a storage vessel 41 can be provided between the pump 40 and the instrument 11 in which plasma-activated liquid can be stored over a predefined or selectable period of time.

The sensor device 36 can be connected with the storage vessel 41 in addition or as an alternative to the circulation 37. Similarly, the storage vessel 41 can be connected with an individual sensor device that is then in turn connected with the control device 19. By control of the pump 40 and/or a not further illustrated pump arranged between the storage vessel 41 and the instrument 11 the control device 19 can define a storage duration for the plasma-treated liquid F. Since the concentration of the species created in the plasma-treated liquid decreases with different rates after plasma treatment of a liquid F, the control device 19 can discharge plasma-treated liquid F by setting a defined storage duration as necessary under control of the sensor device 36 in which, for example, species with short lifetime have mostly disappeared, however, species with long lifetime are contained with higher concentration. On the other hand, if the treatment request comprises predominantly species with short lifetime, it can be determined by means of the sensor device 36 whether these have been created in sufficient concentration in order to supply them immediately to the instrument 11. For this purpose, it can be provided to supply liquid from the storage vessel 41 for post activation to the treatment vessel 16 as necessary. This is indicated in FIG. 1 by double arrows 42, 43.

The device 10 and the instrument 11 described so far operate as follows:

The reservoir vessel 17 is first filled with a liquid to be activated, e.g. sodium chloride solution, via a filler neck 44 (FIG. 1). The control device 19 now activates pump 18 and supplies liquid F in the treatment vessel 16, e.g. in that the treatment vessel 16 is filled with liquid F. In addition, the control device 19 activates the plasma generator 34 and as necessary the gas source 32, such that the plasma applicator 29 now creates a plasma 30 acting on the liquid F (FIG. 2). The sensor device 36 is operating continuously or in discrete intervals in order to detect the quality of the liquid F, i.e. its plasma activation. The quality of the plasma activation is thereby particularly defined by the concentration of selected species, as for example the content of hydronium ions, hydroxide ions, hydrogen peroxide, nitrite, nitrate, peroxynitrite, singlet oxygen, ozone, hydroperoxide, oxygen, superoxide radical ions, hydroxyl radicals and/or hydroperoxyl radicals. For detection of such species the sensor device 36 is configured as so-called pH-single rod measuring cell, as spectroscope for emitted light (IR, visible and/or UV), as absorption spectroscope (as illustrated in FIG. 2), as phosphorescence sensor, for example for radiation of the wave length of 1275 nm for measuring of singlet oxygen or as electron spin resonance spectroscope. The sensor device 36 can also comprise multiple of the indicated measuring devices.

Based on the concentration of the selected species determined by the sensor device, the control device 19 can set, extend or reduce the plasma treatment duration of the liquid, can regulate the parameters of the current or the voltage supplied by the plasma generator 34 and/or can influence the gas flow from the gas source 32. In addition or as an alternative, the control device 19 can set or limit the storage duration of the plasma-treated liquid in the storage vessel 41 and/or can control one or more of the devices 26-28.

The control device 19 can be configured to influence the quality of the liquid F in terms of one or more of the correlations indicated in the following:
Influencing of the effect strength of the plasma, i.e. the electrical power transformed in the plasma or other parameters, depending on the desired pH-value. With a higher power, a lower pH-value is achieved.
Defining the application duration of the plasma on the liquid, depending on the desired pH-value. With a higher application duration, a lower pH-value is achieved.
Defining the effect strength of the plasma, i.e. the electrical power transformed in the plasma or other parameters, depending on the desired ratio of hydrogen to nitrate.
Selection of the liquid to be treated, depending on the desired ratio of hydrogen to nitrate. The liquids provided for selection can be, for example, a NaCl-solution and phosphate buffered NaCl-solution.

The control device 19 can use the chemical and/or physical parameter(s) of the treated liquid F detected by the sensor device 36 in order to control the operation of the plasma application device 25 or the plasma generator 34 in order to achieve a desired quality of the liquid F, i.e. a desired composition of the obtained species. During control of the plasma generator at least one parameter of the current and/or the voltage supplied therefrom is influenced.

With embodiments of the invention a device 10 for creation of a plasma-activated liquid F with defined characteristics is provided. The device 10 comprises a plasma application device 25 provided with a plasma applicator 29, wherein a liquid F is brought into contact with a gas plasma 30 in the plasma application device 25. A sensor device 36 serves for analysis of the composition of the plasma-treated liquid F at least in terms of a species created by the plasma treatment. Based on a concentration of one or more species detected by the sensor device 36, treatment parameters of the liquid F in the plasma application device 25 can be adjusted or modified. Thereby a plasma-treated liquid F with defined characteristics for treatment of a patient is provided.

1. A device for supply of a medical instrument with a plasma-activated liquid, the device comprising:
a plasma generator for supply of a plasma;
a plasma application device in which a liquid can be brought into contact with the plasma and having an outlet in order to discharge liquid from the plasma application device and to supply it to the instrument; and
a sensor device for detection of at least one chemical or physical parameter of the liquid during and/or after the plasma exposure in the plasma application device.

2. The device according to claim 1, wherein the plasma application device comprises a plasma applicator having a gas channel and at least one electrode being in contact with gas from the gas channel, as well as an electrode being in electrical contact with the liquid, both electrodes being connected to the plasma generator.

3. The device according to claim 1, wherein an outlet of the plasma application device is connected to a storage vessel that can be connected with the instrument.

4. The device according to claim 3, wherein the sensor device and/or a control device is connected to at least one control element in order to control the residence time of the liquid in the plasma application device.

5. The device according to claim 1, wherein the plasma generator can be controlled in terms of a supplied voltage, a supplied current, a supplied power, a supplied crest factor or a supplied wave form.

6. The device according to claim 5, wherein the sensor device and/or a control device is connected to the plasma generator in order to control it dependent on the detected parameter.

7. The device according to claim 2, wherein the gas channel is connected to a gas source that can be controlled in terms of the gas flow.

8. The device according to claim 7, wherein the sensor device and/or a control device is connected to the gas source in order to control its gas flow depending on the detected parameter.

9. The device according to claim 1, wherein the sensor device is configured to detect at least one of a temperature, a conductivity, an acidity (pH-value), a chemical composition and a concentration of particular chemical compounds.

10. The device according to claim 1, wherein the sensor device is configured to detect the concentration of plasma created substances in the plasma-activated liquid in the form of at least one of hydronium ions (H₃O⁺), hydroxide ions (OH⁻), hydrogen peroxide (H₂O₂), nitrite ions (NO₂⁻), nitrate ions (NO₃⁻), of hydroxyl radicals (.OH).

11. The device according to claim 1, wherein the sensor device is configured to detect the concentration of plasma created substances in the plasma-activated liquid by spectroscopy.

12. The device according to claim 1, wherein the spectroscopy is absorption spectroscopy of electromagnetic radiation.

13. The device according to claim 1, wherein the sensor device is configured to detect the concentration of plasma created substances in the plasma-activated liquid in form of at least one of singlet oxygen (¹O₂), ozone (O₃), oxygen (O₂), superoxide radical ions (O₂⁻), hydroperoxyl radicals (HOO.), peroxynitrite ions (ONOO⁻) and nitrogen oxides.

14. The device according to claim 1, wherein the sensor device is configured to detect the concentration of plasma created substances in the plasma-activated liquid by means of at least one of phosphorescence or electron spin resonance spectroscopy.

15. A method for providing a plasma-activated treatment liquid, the method comprising:
providing a plasma by a plasma generator;
bringing a liquid into contact with the plasma in a plasma application device and is supplied to an instrument; and
detecting at least one chemical or physical parameter of the liquid during and/or after the plasma exposure in the plasma application device by means of a sensor device, wherein the operation of the plasma generator and/or the residence time of the plasma-treated liquid in a storage vessel is controlled for influencing of the parameter.

16. The method according to claim 15, further comprising:
controlling at least one of a supplied voltage, supplied current, supplied power, supplied crest factor and supplied wave form of the plasma generator.

17. The method according to claim 16, further comprising:
controlling the plasma generator dependent on the detected parameter.

18. The method according to claim 15, further comprising:
controlling a gas flow from a gas source to a gas channel of the plasma application device depending on the detected parameter.

19. The method according to claim 15, further comprising:
detecting with the sensor device at least one of a temperature, a conductivity, an acidity (pH-value), a chemical composition and a concentration of particular chemical compounds.

20. The method according to claim 15, further comprising:
detecting with the sensor device a concentration of plasma created substances in the plasma-activated liquid in the form of at least one of hydronium ions (H₃O⁺) hydroxide ions (OH⁻), hydrogen peroxide (H₂O₂), nitrite ions (NO₂⁻), nitrate ions (NO₃⁻) and hydroxyl radicals (.OH).
